(19) United States
(12) Patent Application Publication
(10) Pub. No.: US 2023/0372092 A1
(43) Pub. Date: Nov. 23, 2023

(54) Fully-Transseptal Cinching Apical Pad

(71) Applicant: Tendyne Holdings, Inc., St. Paul, MN (US)

(72) Inventors: Theodore Paul Dale, Corcoran, MN (US); Tracee Eidenschink, Wayzata, MN (US); Alec King, Maple Grove, MN (US); Chase Carlson, Lakeland, MN (US); David A. Panus, Maple Grove, MN (US); Amy Marie Danielson, St. Paul, MN (US)

(73) Assignee: Tendyne Holdings, Inc., St. Paul, MN (US)

(21) Appl. No.: 18/300,612

(22) Filed: Apr. 14, 2023

Related U.S. Application Data
(60) Provisional application No. 63/342,801, filed on May 17, 2022.

(51) Int. Cl. A61F 2/24

(52) U.S. Cl. CPC A61F 2/2418; A61B 2017/0406

(57) ABSTRACT

An apical pad for securing a prosthetic heart valve within a native valve annulus. The apical pad includes an inner collar having a collapsed condition for transcatheter delivery and an expanded condition arranged to engage an interior surface of a heart wall, an outer collar having a collapsed condition for transcatheter delivery and an expanded condition arranged to engage an exterior of the heart wall, and a connector extending between the inner and outer collars. The inner collar is moveable relative to the outer collar to clamp the heart wall and to seal an incision extending through the heart wall.

### CROSS-REFERENCE TO RELATED APPLICATIONS

This application claims priority to the filing date of U.S. Provisional Patent Application No. 63/342,801, filed May 17, 2022, the disclosure of which is hereby incorporated by reference herein.

### BACKGROUND OF THE DISCLOSURE

The present disclosure relates to collapsible and expandable prosthetic heart valves, and more particularly, to apparatus and methods for stabilizing a collapsible and expandable prosthetic heart valve within a native annulus of a patient.

Prosthetic heart valves that are collapsible to a relatively small circumferential size can be delivered into a patient less invasively than valves that are not collapsible. For example, a collapsible and expandable valve may be delivered into a patient via a tube-like delivery apparatus such as a catheter, a trocar, a laparoscopic instrument, or the like. This collapsibility can avoid the need for a more invasive procedure such as full open-chest, open-heart surgery.

Collapsible and expandable prosthetic heart valves typically take the form of a valve structure mounted on a stent. There are two types of stents on which the valve structures are ordinarily mounted: a self-expanding stent and a balloon-expandable stent. To place such valves into a delivery apparatus and ultimately into a patient, the valve must first be collapsed to reduce its circumferential size.

When a collapsed prosthetic valve has reached the desired implant site in the patient (e.g., at or near the native annulus of the patient's heart valve that is to be repaired by the prosthetic valve), the prosthetic valve can be deployed or released from the delivery apparatus and expanded to its full operating size. For balloon-expandable valves, this generally involves releasing the entire valve, assuring its proper location, and then expanding a balloon positioned within the valve stent. For self-expanding valves, on the other hand, the stent automatically expands as the stent is withdrawn from the delivery apparatus.

The clinical success of collapsible and expandable heart valves is dependent, in part, on the anchoring of the valve within the native annulus. Self-expanding valves typically rely on the radial force exerted by expanding the stent against the native annulus to anchor the prosthetic heart valve. However, if the radial force is too high, the heart tissue may be damaged. If, instead, the radial force is too low, the heart valve may move from its deployed position and/or migrate from the native annulus, for example, into the left ventricle.

Movement of the prosthetic heart valve may result in the leakage of blood between the prosthetic heart valve and the native valve annulus. This phenomena is commonly referred to as paravalvular leakage. In mitral valves, paravalvular leakage enables blood to flow from the left ventricle back into the left atrium during systole, resulting in reduced cardiac efficiency and strain on the heart muscle.

Anchoring prosthetic heart valves within the native valve annulus of a patient, especially within the native mitral valve annulus, can be difficult. For example, prosthetic mitral valves often require a low profile so as not to interfere with atrial function, and the low profile complicates securely anchoring the prosthetic heart valve in place. Moreover, the native mitral valve annulus has reduced calcification or plaque compared to the native aortic valve annulus, for example, which can make for a less stable surface to anchor the prosthetic heart valve. For this reason, collapsible and expandable prosthetic mitral valves often include additional anchoring features such as a tether. The tether is commonly secured to an apical pad that anchors the prosthetic heart valve in position within the native annulus of the patient.

Despite the improvements that have been made to anchoring collapsible and expandable prosthetic heart valves, shortcomings remain. For example, the apical pad is typically inserted through an incision made between the ribs of the patient and secured to an external surface at the apex of the heart. Conventional apical pads therefore require that the incision be of a sufficient size to allow the apical pad to be inserted through the incision before the tether is tensioned and fastened to the apical pad.

### BRIEF SUMMARY OF THE DISCLOSURE

In accordance with a first aspect of the present disclosure, an expandable apical pad is provided. Among other advantages, the apical pad is designed to be collapsed to a delivery condition, loaded within a catheter along with the prosthetic heart valve and delivered to an implant site within the heart before the apical pad is extended through the ventricular wall of the heart, transitioned to an expanded deployed condition and secured to the apex of the heart. As a result, the apical pad disclosed herein may be delivered and secured to the heart in a less invasive manner than apical pads that are not collapsible.

One embodiment of the apical pad includes an inner collar an outer collar and a connector extending between the inner and outer collars. The inner and outer collars each having a collapsed condition for transcatheter delivery and an expanded condition arranged to engage a surface of the heart wall. When in the expanded condition, the inner collar is moveable relative to the outer collar to clamp the heart wall and to seal an incision extending therethrough.

A prosthetic heart valve connectable to an apical pad is also provided herein and includes a prosthetic heart valve having an expandable stent with an inflow end and an outflow end, a valve assembly including a cuff and a plurality of leaflets disposed within the stent, a tether slidingly attached to the stent, and an apical pad. The apical pad including an inner collar arranged to engage an interior surface of a heart wall, an outer collar arranged to engage an exterior surface of the heart wall and a connector provided between the inner and outer collars. When the apical pad is deployed from a delivery device, the inner collar is moveable relative to the outer collar to clamp the heart wall independent of tensioning the tether.

A method of implanting a prosthetic heart valve within a native heart valve annulus is provided herein and includes a method of implanting a prosthetic heart valve in a patient is provided and includes: delivering a delivery device to a target site adjacent to a native valve annulus, the delivery device holding a prosthetic heart valve including a stent, a valve assembly disposed within the stent and a tether attached to the stent and to the apical pad; deploying the prosthetic heart valve from the delivery device within the native valve annulus; creating a passage through the wall of the heart; deploying an outer collar of the apical pad from the delivery device, through the passage and to a location outside the heart; transitioning the outer collar from a delivery condition in which the outer collar has a first cross-section to a deployed condition in which the outer collar has a second cross-section greater than the first cross-section; deploying an inner collar of the apical pad from the delivery device within the heart; transitioning the inner collar from a delivery condition in which the inner collar has a third cross-section to a deployed condition in which the inner collar has a fourth cross-section greater than the third cross-section; and moving the inner collar and the outer toward one another to clamp the heart wall.

### BRIEF DESCRIPTION OF THE DRAWINGS

Various embodiments of the present disclosure are described herein with reference to the drawings, wherein:

FIG. 1 is a highly schematic cutaway view of the human heart, showing two approaches for delivering a prosthetic mitral valve;

FIG. 2 is a highly schematic representation of a native mitral valve and associated cardiac structures;

FIG. 3 is a highly schematic cutaway view of the human heart showing a collapsible and expandable prosthetic heart valve according to the prior art anchored within the native mitral annulus by a tether and an apical pad;

FIG. 4A is a side elevational view of the prosthetic mitral valve of FIG. 3;

FIG. 4B is a plan view of the prosthetic mitral valve of FIG. 3;

FIG. 4C is a side elevational view showing an inner stent disposed within and secured to an outer stent of the prosthetic mitral valve of FIG. 3;

FIG. 5A is a side elevational view of the inner stent of FIG. 4C;

FIG. 5B is a side elevational view of the outer stent of FIG. 4C;

FIGS. 6A and 6B are highly schematic partial views of a delivery catheter deploying the prosthetic mitral valve of FIG. 3;

FIG. 7A is a side elevational view of an apical pad according to a first embodiment of the present disclosure;

FIG. 7B is a side elevational view of an apical pad according to a second embodiment of the present disclosure;

FIG. 7C is a side elevational view of an apical pad according to a third embodiment of the present disclosure;

FIG. 7D is a side elevational view of an apical pad according to a fourth embodiment of the present disclosure;

FIG. 8A is a perspective view of a locking clamp in a locked condition;

FIG. 8B is a perspective view of the locking clamp of FIG. 8A in an unlocked condition;

FIG. 9 is a side elevational view of a modified inner stent according to the present disclosure; and

FIG. 10 is a highly schematic side elevational view showing a delivery catheter loaded with the apical pad of FIG. 7D and secured to a prosthetic mitral valve.

### DETAILED DESCRIPTION

Blood flows through the mitral valve from the left atrium to the left ventricle. As used herein in connection with a prosthetic heart valve, the term “inflow end” refers to the end of the heart valve through which blood enters when the valve is functioning as intended, and the term “outflow end” refers to the end of the heart valve through which blood exits when the valve is functioning as intended. Also as used herein, the terms “substantially,” “generally,” and “about” are intended to mean that slight deviations from absolute are included within the scope of the term so modified.

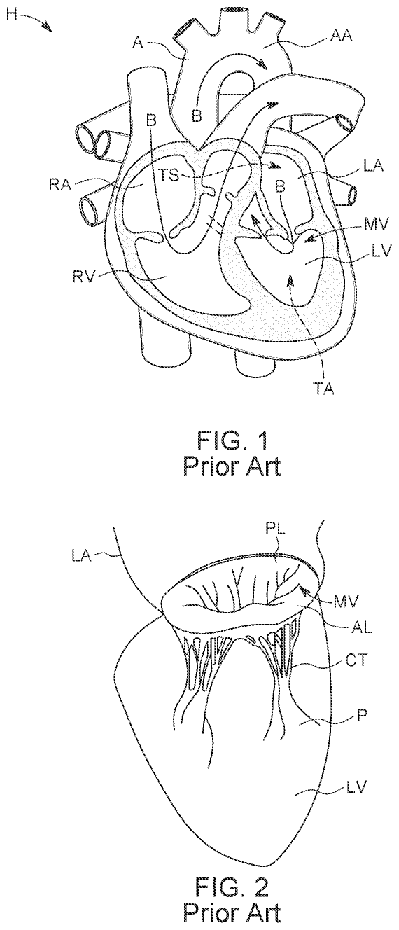

FIG. 1 is a schematic cutaway representation of a human heart H. The human heart includes two atria and two ventricles: right atrium RA and left atrium LA, and right ventricle RV and left ventricle LV. Heart H further includes aorta A and aortic arch AA. Disposed between the left atrium and the left ventricle is mitral valve MV. The mitral valve, also known as the bicuspid valve or left atrioventricular valve, is a dual-flap that opens as a result of increased pressure in left atrium LA as it fills with blood. As atrial pressure increases above that in left ventricle LV, mitral valve MV opens and blood flows into the left ventricle. Blood flows through heart H in the direction shown by arrows “B”.

A dashed arrow, labeled “TA”, indicates a transapical approach of implanting a prosthetic heart valve, in this case to replace the mitral valve. In the transapical approach, a small incision is made between the ribs of the patient and into the apex of left ventricle LV to deliver the prosthetic heart valve to the target site. A second dashed arrow, labeled “TS”, indicates a transseptal approach of implanting a prosthetic heart valve in which the valve is inserted into the femoral vein and passed through the septum between right atrium RA and left atrium LA. Other approaches for implanting a prosthetic heart valve are also possible and may be used to implant the collapsible prosthetic heart valve described in the present disclosure.

FIG. 2 is a more detailed schematic representation of native mitral valve MV and its associated structures. As previously noted, mitral valve MV includes two flaps or leaflets, posterior leaflet PL and anterior leaflet AL, disposed between left atrium LA and left ventricle LV. Cord-like tendons, known as chordae-tendineae CT, connect the two leaflets to the medial and lateral papillary muscles P. During atrial systole, blood flows from higher pressure in left atrium LA to lower pressure in left ventricle LV. When left ventricle LV contracts during ventricular systole, the increased blood pressure in the chamber pushes the posterior and anterior leaflets to close, preventing the backflow of blood into left atrium LA. Since the blood pressure in left atrium LA is much lower than that in left ventricle LV, the leaflets attempt to evert to low pressure regions. Chordae tendineae CT prevent the eversion by becoming tense, thus pulling on the leaflets and holding them in the closed position.

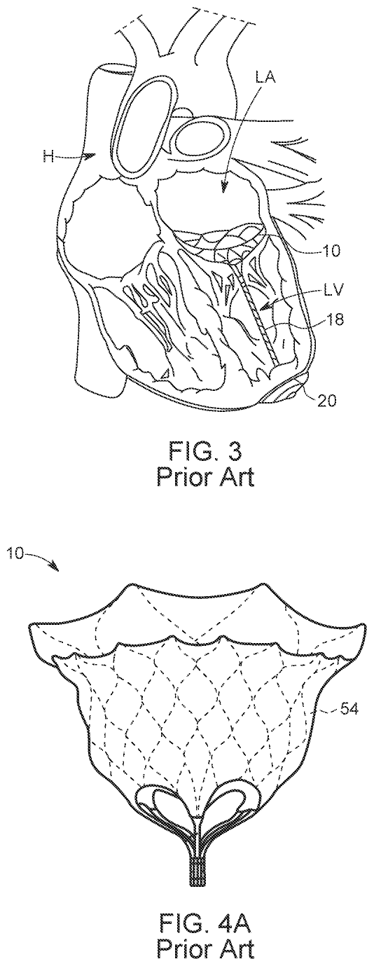

FIG. 3 illustrates a known collapsible and expandable prosthetic heart valve 10 secured within the native mitral valve annulus of a patient. For balloon-expandable variants, prosthetic heart valve 10 may be expandable, but not collapsible, or not readily collapsible, once expanded. When used to replace native mitral valve MV, prosthetic valve 10 may have a low profile so as not to interfere with the heart's electrical conduction system pathways or the atrial function.

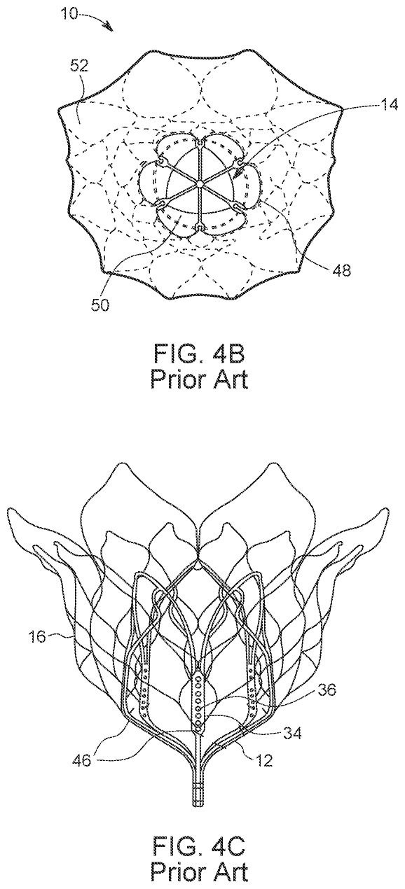

With continued reference to FIG. 3 and further reference to FIGS. 4A-4C, prosthetic heart valve 10 includes an inner stent 12 securing a valve assembly 14, an outer stent 16 secured to and disposed around the inner stent and a tether 18 configured to be secured to an apical pad 20. Both the inner stent 12 and the outer stent 16 may be formed from biocompatible materials that are capable of self-expansion, for example, shape-memory alloys such as nitinol. Alternatively, inner stent 12 or outer stent 16 may be balloon expandable or expandable by another force exerted radially outward on the stent. When expanded, outer stent 16 may exert an outwardly directed radial force against the native valve annulus that assists in anchoring inner stent 12 and valve assembly 14 within the native annulus.

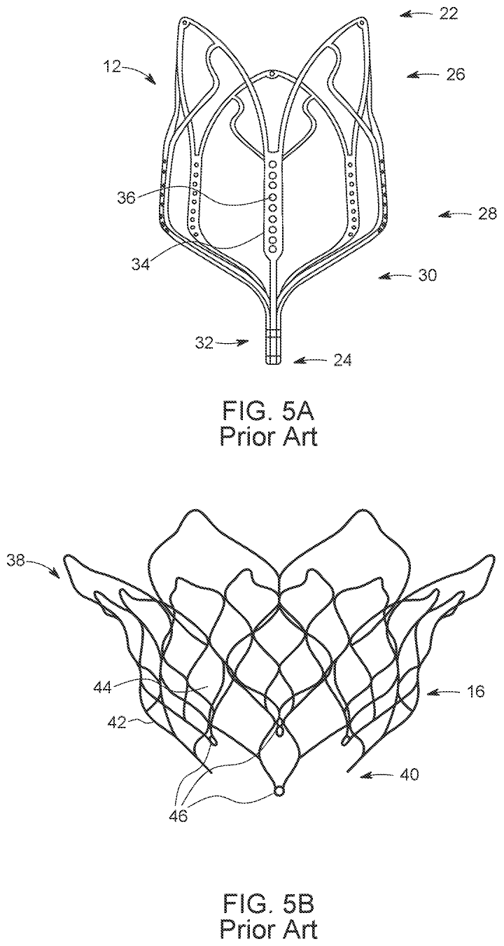

Referring to FIG. 5A, inner stent 12 extends along a longitudinal axis between an inflow end 22 and an outflow end 24. In one example, inner stent 12 is formed by laser cutting a predetermined pattern into a metallic tube, such as a self-expanding tube formed of a shape-memory alloy such as nitinol, to form four portions: cusps 26, a body portion 28, a strut portion 30 and a tether clamp 32 that clamps and secures tether 18 (shown in FIG. 3).

Strut portion 30 may include, for example, six struts that extend radially inward from body portion 28 to tether clamp 32. When inner stent 12 is expanded, strut portion 30 forms a radial transition between body portion 28 and tether clamp 32 that facilitates crimping of the inner stent when tether 18 is retracted within a delivery device. Body portion 28 may also include six longitudinal posts 34 having a plurality of bores or eyelets 36 for securing valve assembly 14 to the inner stent 12 by one or more sutures. As shown in FIG. 5A, three cusps 26 are positioned at the inflow end 22 of inner stent 12. Each of cusps 26 is circumferentially disposed between a pair of adjacent longitudinal posts 34.

Outer stent 16, shown in FIG. 5B, extends along the longitudinal axis between an atrial end 38 and a ventricular end 40. Outer stent 16 may include a plurality of struts 42 that form cells 44 extending about the outer stent in one or more annular rows. In one example, outer stent 16 is formed by laser cutting a predetermined pattern into a metallic tube, such as a self-expanding nitinol tube. Cells 44 may be substantially the same size around the perimeter of stent 16 and along the length of the stent. Alternatively, cells 44 near the atrial end 38 of outer stent 16 may be larger than the cells near the ventricular end 40. When outer stent 16 is expanded, the struts 42 forming a cell 44 in the annular row of cells adjacent the atrial end 38 of the outer stent may bend about the midsection of the cell (e.g., in a direction generally orthogonal to the longitudinal axis) such that the upper apex of the cell extends radially outward relative to the midsection of the cell and forms an atrial flange. When prosthetic valve 10 is disposed within a native mitral valve MV, the flange is designed to protrude into the left atrium LA and engage an upper surface of the native mitral valve annulus to form a seal and prevent blood from flowing between the prosthetic valve and the native annulus.

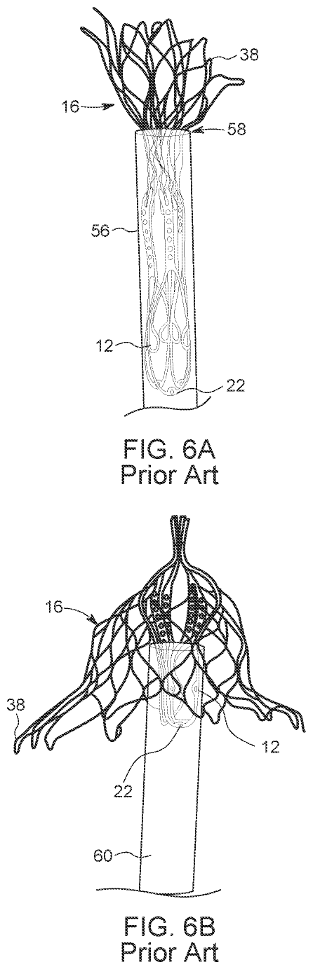

A plurality of attachment features 46 may lie at the intersections of the struts 42 that form the cells 44 at the ventricular end 40 of outer stent 16. Attachment features 46 may include an eyelet that facilitates the suturing of outer stent 16 to the longitudinal posts 34 of inner stent 12 thereby securing the inner and outer stents together as shown in FIG. 4C. In one example, each attachment feature 46 may be sutured to a single eyelet 36 of longitudinal post 34, proximate to the outflow end 24 of inner stent 12 such that outer stent 16 may evert relative to and about the inner stent as is described in more detail below with reference to FIGS. 6A and 6B.

Referring back to FIGS. 4A and 4B, valve assembly 14 may be secured to inner stent 12 by suturing the valve assembly to longitudinal posts 34. Valve assembly 14 includes a cuff 48 and a plurality of leaflets 50 that open and close collectively to function as a one-way valve. The eyelets 36 of longitudinal posts 34 facilitate the suturing of the leaflet commissure to the body portion 28 of inner stent 12. Cuff 48 and leaflets 50 may be wholly or partly formed of any suitable biological material, such as bovine or porcine pericardium, or biocompatible polymer, such as polytetrafluorethylene (PTFE), urethanes and the like.

An inner skirt 52 may be disposed on a luminal surface of outer stent 16. Inner skirt 52 may also be formed of any suitable biological material, such as bovine or porcine pericardium, or biocompatible polymer, such as PTFE, urethanes or similar materials. An outer skirt 54 may be disposed about an abluminal surface of outer stent 16. Outer skirt 54 may be formed of a polyester fabric that promotes tissue ingrowth.

Prosthetic heart valve 10 may be used to repair a malfunctioning native heart valve, such as a native mitral valve, or a previously implanted and malfunctioning prosthetic heart valve. In embodiments in which outer stent 16 is designed to evert, prosthetic heart valve 10 may be collapsed and loaded within a delivery device 56 such that the atrial end 38 of outer stent 16 faces a leading end 58 of the delivery device and the inflow end 22 of inner stent 12 faces a trailing end (not shown) of the delivery device as shown in FIG. 6A. Delivery device 56 may then be percutaneously introduced into the patient and delivered to a desired implant site at or near the native mitral annulus.

Once delivery device 56 has reached the target site, a physician may unsheathe prosthetic heart valve 10 to first allow the outer stent 16 to expand from the collapsed condition and evert about inner stent 12 as the outer stent expands and engages the native valve annulus. Further unsheathing of prosthetic heart valve 10 will allow the inner stent 12 to expand from the collapsed condition to the expanded condition within the anchored outer stent 16 and allow the leaflets 50 to act as a one-way valve. The physician may then make an incision between the ribs of the patient and into the apex of left ventricle LV. After the incision has been made, tether 18 may be pulled through the incision so that the tether extends out from the left ventricle LV of the heart. Apical pad 20 may then be inserted through the incision and placed against an external surface of the apex before the tether is tensioned and secured to the apical pad as shown in FIG. 3. It will be appreciated that the incision must be of sufficient size to allow apical pad 20 to be inserted through the incision and fastened to the heart to anchor prosthetic valve 10 within the native valve annulus. An apical pad that can be secured to the heart through smaller incisions to reduce the invasiveness of the valve repair procedure is desired.

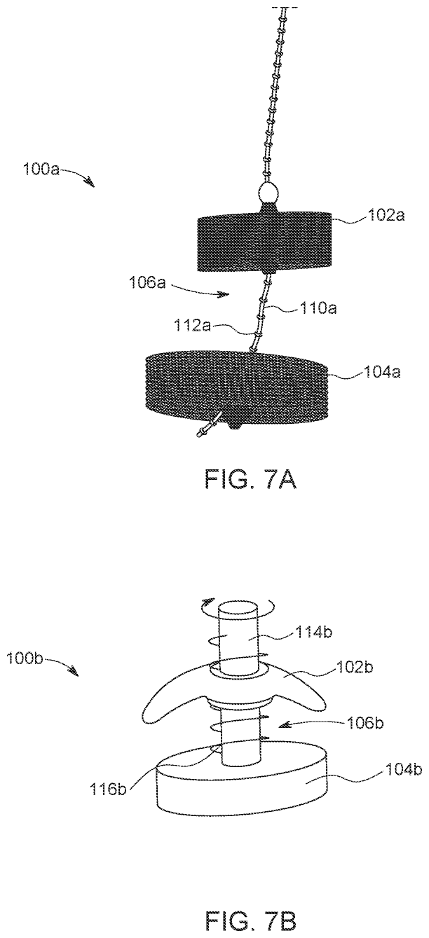

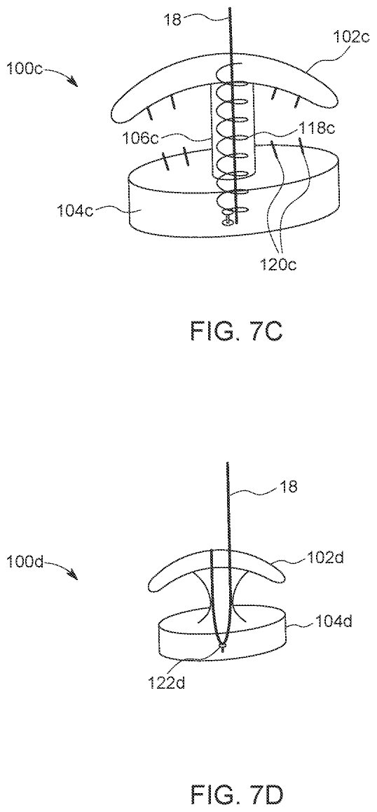

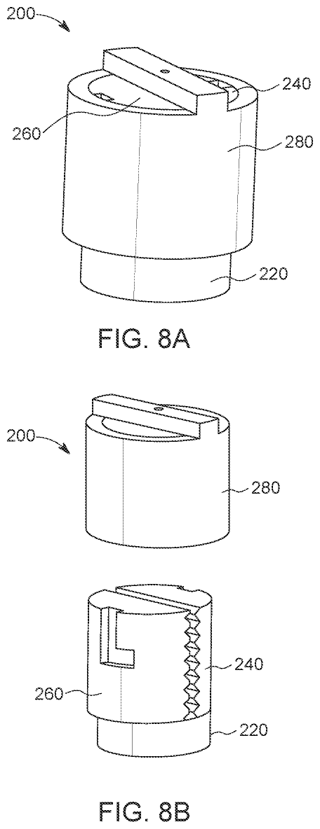

FIGS. 7A-7D illustrate collapsible and expandable apical pads 100a-100d according to various embodiments of the present disclosure. Each one of apical pads 100a-100d is radially collapsible to a delivery condition in which the apical pad can be loaded within a delivery device and percutaneously delivered to the heart of a patient and then expanded to a deployed condition in which the apical pad may be fastened to the apex of the heart. The collapsibility of apical pad 100a-100d allows it to be delivered into the body of a patient in a less invasive manner than conventional apical pad 20 (described above with reference to FIG. 3). In certain aspects, apical pad 100a-100d may be used in conjunction with a locking clamp 200 (shown in FIGS. 8A and 8B) that slidingly couples tether 18 to the inner stent. Put another way, locking clamp 200 is transitionable between an unlocked condition in which tether 18 is slidable relative to the locking clamp and a locked condition in which the tether is fixed to the locking clamp. As a result, apical pad 100a-100d may be clamped to the myocardium of the heart before the tether is tensioned. Clamping apical pad 100a-100d to the myocardium of the heart and tensioning the tether in separate and independent steps affords a physician with greater procedural flexibility including the option to abandon the apical pad in the event that complications arise during the procedure. Although apical pad 100a-100d and locking clamp 200 are described herein as replacements for apical pad 20 and clamp 32, respectively, it will be appreciated that apical pad 100a-100d and locking clamp 200 may be attached to the tether of any prosthetic heart valve and used in the repair of the native mitral valve of a patient or another cardiac valve such as the aortic valve.

Apical pads 100a-100d share several common features. The features shared among apical pads 100a-100d will be described first and then features unique to each embodiment will be described thereafter. Referring to FIGS. 7A-7D, each apical pad 100a-100d includes an inner collar 102a-102d, an outer collar 104a-104d and a connector 106a-106d extending between the inner and outer collars. In one aspect, apical pad 100a-100d is formed by laser cutting a predetermined pattern into a metallic tube formed of biocompatible materials capable of self-expansion, for example, shape-memory alloys such as nitinol. However, the apical pads 100a-100d may be formed via other methods, including for example braiding strands of wire, such as Nitinol, into a mesh structure. The mechanical properties of the shape-memory alloys provide inner collar 102a-102d and outer collar 104a-104d the ability to compress and expand, thus, transitioning apical pad 100a-100d between a collapsed condition (e.g., a delivery condition) and a radially expanded condition (e.g., a deployed condition). The inner collar 102a-102d and the outer collar 104a-104d of apical pad 100a-100d may be heat-set, or otherwise preset, to the radially expanded condition. Thus, once apical pad 100a-100d is released from a delivery device, such as delivery device 56, inner collar 102a-102d and outer collar 104a-104d will expand to their preset configuration and cause the apical pad to transition to its deployed configuration.

When in the radially expanded configuration, the inner collar 102a-102d and the outer collar 104a-104d are generally disc shaped. The surface of inner collar 102a-102d that faces outer collar 104a-104d may be convexly contoured to match the anatomy of the inner wall at the apex of the heart. Similarly, the surface of outer collar 104a-104d that faces inner collar 102a-102d may be concavely contoured to match the anatomy of the outer wall at the apex of the heart. The convex surface of the inner collar 102a-102d and the concave surface of outer collar 104a-104d are sometimes referred to herein as the “clamping surfaces” because they clamp the myocardium to secure the apical pad to the heart. A padding may be sewn to or otherwise coupled to the clamping surfaces of inner collar 102a-102d and outer collar 104a-104d. The padding may be formed from a polyester fabric or another material that facilitates the clotting of blood and the ingrowth of tissue when apical pad 100a-100d is clamped to the myocardium.

Apical pad 100a-100d has a cross-section that is approximately equal to or less than 24 French (8 mm), and preferably equal to or less than 18 French (6 mm), when the apical pad is in the delivery (e.g. collapsed or unexpanded) condition. In this manner, apical pad 100a-100d can be loaded within a delivery device sized for transseptal delivery through the femoral artery. When inner collar 102a-102d and outer collar 104a-104d are expanded in the radial direction, apical pad 100a-100d may have a cross-section that is greater than 8 mm.

Features unique to each apical pad 100a-100d will now be described. With reference to FIG. 7A, a cinch cord 110a is disposed within a channel extending through the inner collar 102a, connector 106a, and the outer collar 104a of the apical pad. Cinch cord 110a includes a series of locking features 112a disposed at intervals along the length of the cinch cord. Example locking features 112a include beads and notches. It will be appreciated that the nitinol struts forming connector 106a are not illustrated in FIG. 7A in order to more clearly illustrate cinch cord 110a and locking features 112a.

The locking features 112a of cinch cord 110a are designed to interact with a protrusion (not shown) that extends into the channel of inner collar 102a in a manner that allows the inner collar to slide over the cinch cord in a direction toward outer collar 104a but prevents the inner collar from sliding over the cinch cord in a direction away from the outer collar. The projection of the protrusion creates a small gap between the free end of the protrusion and an inner wall of the inner collar defining the channel. The protrusion may have a first angled surface and a second non-angled surface. The first angled surface may be angled toward cinch cord 110a and away from the clamping surface of inner collar 102a. Put differently, the first angled surface may be angled toward the inner surface of the inner collar. The angled surface of the protrusion may be formed from a resilient material designed to flex (e.g., compress) when subjected to a force. Thus, when inner collar 102a is slid toward outer collar 104a, and locking feature 112a engages the angled surface of the protrusion, the angled surface compressed and expands the gap to allow the locking feature 112a of cinch cord 110a to be slid through the expanded gap. Conversely, the second non-angled surface extends generally perpendicular to an axis of the channel and is formed from a rigid material. When inner collar 102a is urged away from outer collar 104a, locking feature 112a engages the rigid surface of the protrusion and prevents the locking feature from sliding through the gap which, in turn, prevents the inner collar from moving away from the outer collar. These locking features 112a may allow for the inner collar 102a to be pulled toward the outer collar 104a (or vice versa) to allow for adjustable clamping tension. In other words, for the same anatomical wall thickness, a relatively large distance between the two collars may provide less clamping force than a relatively small distance. Similarly, a patient with a ventricle wall having a large thickness may be clamped with the same force as a patient with a ventricle wall having a smaller thickness by adjusting the distance between the collars accordingly.

Referring to FIG. 7B, the connector 106b of apical pad 100b is formed as an elongate post 114b having a first helical thread 116b. A corresponding second helical thread (not shown) is provided on an inner wall of inner collar 102b. Rotation of inner collar 102b in a first direction (e.g., clockwise) relative to elongate post 114b will thus cause the inner collar to be threaded along the elongate post towards outer collar 104b. On the other hand, rotation of inner collar 102b in an opposite direction (e.g., counterclockwise) relative to the elongate post will result in the inner collar moving away from the outer collar. It will be understood that the relative rotational movement between inner collar 102b and elongate post 114b may be caused by rotating the inner collar while holding the elongate post stationary, or alternatively, by rotating the elongate post while holding the inner collar stationary.

As shown in FIG. 7C, the connector 106c includes a spring 118c that biases inner collar 102c away from outer collar 104c. The length of the spring 118c may be slightly greater than the thickness of the heart wall. Thus, when inner collar 102c and outer collar 104c are deployed from a delivery device, such as delivery device 56, spring 118c assists in spacing the inner collar away from the outer collar and positioning the inner and outer collars on opposite sides of the heart wall. The clamping surfaces of inner collar 102c and outer collar 104c include anchoring features 120c such as spikes, barbs and the like designed to anchor the inner and outer collars into tissue.

Turning now to FIG. 7D, the outer collar 104d of apical pad 100d includes a beam extending generally orthogonal to the longitudinal axis the apical pad and across the channel extending through the outer collar. A pulley 122d or similar device, such as a round edge eyelet, configured to receive tether 18, or the tether of an alternate embodiment of a prosthetic heart valve, is secured to the beam. Tether 18 may be fixed to inner collar 102d, threaded through the channel of connector 106d, wrapped around pulley 122d and threaded back through the channel and the inner collar such that the inner collar and the outer collar 104b of apical pad 100d will be cinched together when the tether is tensioned. The connector 106d of apical pad 100d may be formed of nitinol struts to temporarily separate inner collar 102d and outer collar 104d during delivery and before the tether is tensioned.

FIGS. 8A and 8B are perspective views of a tether locking clamp 200 in an unlocked condition and a locked condition, respectively. Locking clamp 200 is designed to secure tether 18 to prosthetic heart valve 10 after tensioning the tether following delivery of the prosthetic heart valve and apical pad 100a-100d using a transseptal approach, in which the apical pad and the tether are fixed together, and the tether is only fixed to inner stent 12 after the apical pad is deployed and clamped to the heart. However, it should be understood that locking clamp 200 may also be suitable for locking tether 18 at a desired tension using other approaches, such as a transapical approach.

Locking clamp 200 includes a base 220, a first clamp portion 240, a second clamp portion 260, and a cuff 280. Tether 18 may be threaded through an aperture in base 220 and an aperture in cuff 280. First clamp portion 240 and second clamp portion 260 are configured to come together and clamp the tether after the tether has been tensioned thereby fixing the tether at the desired tension. Cuff 280 defines a cavity to receive first clamp portion 240 and second clamp portion 260 urge the first and second clamp portions towards one another to secure tether 18 after the tether has been tensioned.

When replacing clamp 32 with locking clamp 200, inner stent 12 may be modified as shown in FIG. 9. Specifically, the six struts of strut portion 30 may be bent towards the inflow end 22 of inner stent 12 and secured together at a junction by locking clamp 200. Bending the struts of strut portion 30 toward the inflow end 22 of inner stent 12 allows a surgeon performing a heart valve replacement procedure using a transseptal approach improved access to locking clamp 200 to facilitate transitioning the locking clamp from the unlocked to locked configurations.

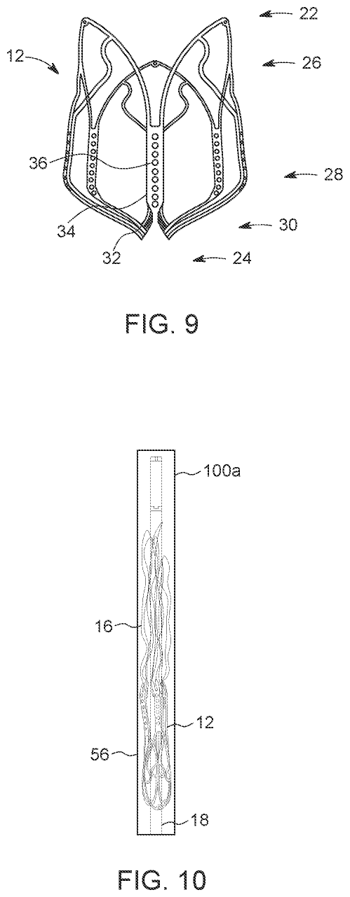

Use of apical pad 100a to anchor prosthetic heart valve 10 within a native mitral valve annulus will now be described with reference to FIGS. 7A-10. With a first end of tether 18 secured to apical pad 100a, a physician may take the free end of the tether and inert it through an aperture in the base 220 and an aperture the cuff 280 of locking clamp 200 to slidingly couple the apical pad and prosthetic heart valve 10 to one another. The coupled prosthetic heart valve 10 and elongated apical pad 100a may then be loaded into delivery device 56. More particularly, outer stent 16 may be everted and prosthetic heart valve 10 may be collapsed and loaded within delivery device 56 such that the atrial end 38 of outer stent 16 faces the leading end 58 of the delivery device and the inflow end 22 of inner stent 12 faces the trailing end of the delivery device. Apical pad 100a may then be transitioned to the delivery condition and loaded into the leading end 58 of delivery device 56 such that the apical pad is disposed between prosthetic heart valve 10 and the leading end of the delivery device with the free end of the tether extending back towards the trailing end of the delivery device.

Delivery device 56 may be percutaneously introduced into the patient, for example, via the femoral vein and delivered into the left ventricle LV using a transseptal approach. With the leading end 58 of delivery device 56 positioned within the left ventricle LV, the physician may use the leading end of the delivery device (or a separate cutting tool) to puncture through the myocardium at the apex of the heart. A plunger (not shown) may then be used to push the outer collar 104a of apical pad 100a out from the leading end 58 of delivery device 56 and through the wall of the heart. The outer collar 104a of apical pad 100a may then naturally expand in the radial direction to its preset condition. The leading end 58 of delivery device 56 may then be retracted to a location within the left ventricle and the plunger may be used to push the inner collar 102a of apical pad 100a out from the leading end of the delivery device 56 allowing the inner collar to radially expand to its preset condition as shown in FIG. 7A. The physician may then use the leading end 58 of delivery device 56, or another tool, to apply a force to expanded inner collar 102a and urge the inner collar towards outer collar 104a. As inner collar 102a slides along cinch cord 110a toward outer collar 104a, the locking feature 112a of the cinch cord compresses the angled surface of the protrusion and allows the locking features to slide over the protrusion, thereby cinching the inner collar towards the outer collar and clamping the inner and outer collars to the myocardium. The padding provided on the clamping surfaces of inner collar 102a and outer collar 104a seals the puncture when apical pad 100a is clamped to the heart wall.

The leading end 58 of delivery device 56 may then be retracted to a location within the left atrium and adjacent to the native mitral valve annulus. Once the leading end 58 of delivery device 56 has been properly positioned, the physician may unsheathe prosthetic heart valve 10 allowing outer stent 16 to evert about inner stent 12 within left atrium LA. The physician may then move the leading end 58 of delivery device 56 to a location within the native mitral valve annulus until the outer stent is properly positioned within the native valve annulus and the flange of the outer stent is engaged with an atrial side of the native annulus. Further unsheathing of prosthetic heart valve 10 will cause the inner stent 12 to expand from the collapsed condition to the expanded condition, within anchored outer stent 16, and allow the leaflets 50 to act as a one-way valve. After the physician has confirmed that prosthetic heart valve 10 has been properly positioned, and leaflets 50 are properly coapting, the physician may tension tether 18 by pulling the free end of tether 18 towards the trailing end of delivery device 56. When tether 18 has been properly tensioned, the cuff 280 of locking clamp 200 may then be slid over the first clamp portion 220 and the second clamp portion 240 of the locking cuff, urging the first and second clamp portions towards one another to clamp the tether at the desired tension and secure prosthetic heart valve 10 within the native mitral annulus.

A prosthetic heart valve coupled to apical pads 100b-100d may be implanted within the native mitral annulus as described above but for the manner in which the apical pads are clamped to the myocardium. For this reason, only the step of clamping apical pads 100b-100d to the heart wall will be described hereinafter.

Referring to FIG. 7B, apical pad 100b may be clamped to the heart by rotating inner collar 102b along the helical thread 116b of post 114b. Rotating inner collar 102b in this manner will thread the inner collar along the post towards the outer collar 104b will clamp the inner and outer collars to the myocardium.

With reference to FIG. 7C, a physician may clamp apical pad 100c to the heart by retracting tether 18 towards the trailing end of delivery device 18 causing the clamping surface of outer collar 104c to engage the exterior surface of the heart wall and applying a force to the inner collar 102c of the apical pad 100c, using the leading end 58 of delivery device 56 (or another tool), to overcome the biasing force of spring 118c thereby causing the clamping surface of the inner collar to engage the interior surface of the heart wall. As spring 118c compresses, the anchoring features 120c on inner collar 102c and outer collar 104c will puncture through the myocardium and secure apical pad 100c to the apex of the heart.

Turning now to FIG. 7D, apical pad 100d may be secured to the apex of the heart by pulling the free end of tether 18 towards the trailing end of delivery device 56 causing the inner collar 102d and the outer collar 104d to cinch toward one another and to clamp the myocardium. Once the tether has been tensioned to the desired tension, the cuff 280 may then be slid over the first clamp portion 220 and the second clamp portion 240 of locking clamp 200 to prevent the inner collar 102d and the outer collar 104d from subsequently being separated.

It will be appreciated that because apical pads 100a-100d are delivered using a transseptal approach, no incision between the patient's ribs need to be made. As a result, apical pad 100 allows for a less invasive valve repair procedure than apical pad 20.

To summarize the foregoing, an apical pad includes an inner collar having a collapsed condition for transcatheter delivery and an expanded condition arranged to engage an interior surface of a heart wall, an outer collar having a collapsed condition for transcatheter delivery and an expanded condition arranged to engage an exterior of the heart wall; and a connector extending between the inner and outer collars, wherein the inner collar is moveable relative to the outer collar to clamp the heart wall and to seal an incision extending therethrough; and/or
the connector may include a cinch cord having one-way locking features disposed at intervals along a length of the cinch cord; and/or
the connector may comprises a post having a first thread arranged to engage a second thread on the inner collar to threadingly advance the inner collar along the post toward the outer collar; and/or
the connector may include a spring; and/or
the outer collar of the apical pad may be secured to a tether having a free end, the free end of the tether extending through the inner collar; and/or
the outer collar may include a pulley; and/or
the inner collar may be secured to a tether having a free end, the free end of the tether may be wrapped around the pulley and extend back through the inner collar such that tensioning the tether will move the inner and outer collars toward one another; and/or
the connector may further include a resilient material to temporarily separate the inner and outer collars until the tether is tensioned.

In another embodiment, a prosthetic heart valve includes an expandable stent having an inflow end and an outflow end; a valve assembly disposed within the stent, the valve assembly including a cuff and a plurality of leaflets; a tether slidingly attached to the stent; and an apical pad attached to the tether, the apical pad including an inner collar arranged to engage an interior surface of a heart wall, an outer collar arranged to engage an exterior surface of the heart wall and a connector provided between the inner and outer collars, wherein the inner collar is moveable relative to the outer collar to clamp the heart wall independent of tensioning the tether; and/or
the connector may include a cord having one-way locking features disposed at intervals along a length of the cord; and/or
the connector may include a post having a first helical thread arranged to engage a second helical thread on the inner collar; and/or
the connector may include a spring, and the tether may be secured to the outer collar and have a free end that extends through the inner collar; and/or
the outer collar may include a pulley, and the tether may secured to the inner collar, wrapped around the pulley and extend back through the inner collar such that tensioning the tether will move the inner and outer collars toward one another.

In another embodiment, a method of implanting a prosthetic heart valve in a patient is provided and includes: delivering a delivery device to a target site adjacent to a native valve annulus, the delivery device holding a prosthetic heart valve including a stent, a valve assembly disposed within the stent and a tether attached to the stent and to the apical pad; deploying the prosthetic heart valve from the delivery device within the native valve annulus; creating a passage through the wall of the heart; deploying an outer collar of the apical pad from the delivery device, through the passage and to a location outside the heart; transitioning the outer collar from a delivery condition in which the outer collar has a first cross-section to a deployed condition in which the outer collar has a second cross-section greater than the first cross-section; deploying an inner collar of the apical pad from the delivery device within the heart; transitioning the inner collar from a delivery condition in which the inner collar has a third cross-section to a deployed condition in which the inner collar has a fourth cross-section greater than the third cross-section; and moving the inner collar and the outer toward one another to clamp the heart wall; and/or
the method may further include tensioning the tether after the moving step; and/or
when the apical pad is in the delivery condition, the apical pad may have a cross-section approximately equal to or less than 24 French (8 mm) and when the apical pad is in the deployed condition, the apical pad may have a cross-section greater than 8 mm; and/or
the apical pad may include a cinch and the moving step may further include: sliding the inner collar along the cinch toward the outer collar; and/or
the apical pad may include a post having a thread and the moving step may further include: threading the inner collar along the thread of the post to advance the inner collar along the post and toward the outer collar; and/or
the apical pad may include a spring and the may tether is secured to the outer collar, and the moving step may further include: tensioning the tether and pushing the inner collar toward the outer collar; and/or
the outer collar may include a pulley and the tether may be secured to the inner collar, wrapped around the pulley, and passed back through the inner collar and the moving step may further include: tensioning the tether to move the inner and outer collars toward one another.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims. For example, while the foregoing disclosure describes the struts forming an ellipse in the deployed condition, that is not necessarily the case. The struts in the deployed condition can form any number of shapes depending on the total number of struts in the apical pad and the angle between the first and second ends of the struts.

1. An apical pad, comprising:
an inner collar having a collapsed condition for transcatheter delivery and an expanded condition arranged to engage an interior surface of a heart wall;
an outer collar having a collapsed condition for transcatheter delivery and an expanded condition arranged to engage an exterior of the heart wall; and
a connector extending between the inner and outer collars,
wherein the inner collar is moveable relative to the outer collar to clamp the heart wall and to seal an incision extending therethrough.

2. The apical pad of claim 1, wherein the connector includes a cinch cord having one-way locking features disposed at intervals along a length of the cinch cord.

3. The apical pad of claim 1, wherein the connector comprises a post having a first thread arranged to engage a second thread on the inner collar to threadingly advance the inner collar along the post toward the outer collar.

4. The apical pad of claim 1, wherein the connector includes a spring.

5. The apical pad of claim 4, further comprising a tether secured to the outer collar, the tether having a free end extending through the inner collar.

6. The apical pad of claim 1, wherein the outer collar includes a pulley or a round edge eyelet.

7. The apical pad of claim 6, further comprising a tether secured to the inner collar, wrapped around the pulley or round edge eyelet, and extending back through the inner collar such that tensioning the tether will cinch the inner and outer collars toward one another.

8. The apical pad of claim 7, wherein the connector further includes a resilient material to temporarily separate the inner and outer collars until the tether is tensioned.

9. A prosthetic heart valve, comprising:
an expandable stent having an inflow end and an outflow end;
a valve assembly disposed within the stent, the valve assembly including a cuff and a plurality of leaflets;
a tether slidingly attached to the stent; and
an apical pad attached to the tether, the apical pad including an inner collar arranged to engage an interior surface of a heart wall, an outer collar arranged to engage an exterior surface of the heart wall and a connector provided between the inner and outer collars,
wherein the inner collar is moveable relative to the outer collar to clamp the apical pad to the heart wall independent of tensioning the tether.

10. The prosthetic heart valve of claim 9, wherein the connector includes a cord having one-way locking features disposed at intervals along a length of the cord.

11. The prosthetic heart valve of claim 9, wherein the connector comprises a post having a first helical thread arranged to engage a second helical thread on the inner collar.

12. The prosthetic heart valve of claim 9, wherein the connector includes a spring, and the tether is secured to the outer collar and has a free end that extends through the inner collar.

13. The prosthetic heart valve of claim 9, wherein the outer collar includes a pulley, and the tether is secured to the inner collar, wrapped around the pulley and extends back through the inner collar such that tensioning the tether will move the inner and outer collars toward one another.

14. A method of implanting a prosthetic heart valve in a patient, the method comprising:
delivering a delivery device to a target site adjacent to a native valve annulus, the delivery device holding a prosthetic heart valve including a stent, a valve assembly disposed within the stent and a tether attached to the stent and to the apical pad;
deploying the prosthetic heart valve from the delivery device within the native valve annulus;
creating a passage through the wall of the heart;
deploying an outer collar of the apical pad from the delivery device, through the passage and to a location outside the heart;
transitioning the outer collar from a delivery condition in which the outer collar has a first cross-section to a deployed condition in which the outer collar has a second cross-section greater than the first cross-section;
deploying an inner collar of the apical pad from the delivery device within the heart;
transitioning the inner collar from a delivery condition in which the inner collar has a third cross-section to a deployed condition in which the inner collar has a fourth cross-section greater than the third cross-section; and
moving the inner collar and the outer toward one another to clamp the heart wall.

15. The method of claim 14, further comprising tensioning the tether after the moving step.

16. The method of claim 14, wherein when the apical pad is in the delivery condition, the apical pad has a cross-section approximately equal to or less than 24 French (8 mm) and when the apical pad is in the deployed condition, the apical pad has a cross-section greater than 8 mm.

17. The method of claim 14, wherein the apical pad comprises a cinch and the moving step further comprises:
sliding the inner collar along the cinch toward the outer collar.

18. The method of claim 14, wherein the apical pad comprises a post having a thread and the moving step further comprises:
threading the inner collar along the thread of the post to advance the inner collar along the post and toward the outer collar.

19. The method of claim 14, wherein the apical pad comprises a spring and the tether is secured to the outer collar, and the moving step further comprises:
tensioning the tether pushing the inner collar toward the outer collar.

20. The method of claim 15, wherein outer collar comprises a pulley and the tether is secured to the inner collar, wrapped around the pulley, and passed back through the inner collar and the moving step further comprises:
tensioning the tether to move the inner and outer collars toward one another.
